(19) United States
(12) Patent Application Publication
(10) Pub. No.: US 2009/0104690 A1
(43) Pub. Date: Apr. 23, 2009

(54) DEVICE FOR DETECTING AN ENZYME IN A SAMPLE

(76) Inventors: Simon W. Bayliff, North Yorkshire (GB); Alicia Essler, North Yorkshire (GB); David Greenhalgh, North Yorkshire (GB); Maurice C. Biott, Silsden Keighley West Yorkshire (GB)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/814,807

(22) PCT Filed: Jan. 27, 2006
(86) PCT No.: PCT/GB2006/000283

§ 371 Date: Aug. 12, 2008

(30) Foreign Application Priority Data
Jan. 28, 2005 (GB) 0501818.9

(51) Int. Cl. C12M 1/00

(52) U.S. Cl. 435/287.1

(57) ABSTRACT

A device for detecting a mammalian host-derived enzyme in a sample of a wound fluid, said device comprising: a housing having an inlet for the sample and side walls defining a fluid flow path extending from said inlet an indicator moiety that is bound to a solid substrate by means of a peptide linker moiety that is cleavable by said host-derived enzyme, said solid substrate being located in a reaction zone of said fluid flow path; and a detector moiety located in a detection zone downstream from the reaction zone in said fluid flow path, wherein the detector moiety interacts with an indicator moiety that has been cleaved from said solid substrate to produce a detectable change in said detection zone. Suitably, the device further comprises a control moiety located in a control zone in said device, wherein the control moiety can interact with a component of the wound fluid sample to improve the accuracy of the device. Also provided are systems comprising a device of the invention and a swab or biopsy punch adapted for use therewith.

The present invention relates to diagnostic devices for detecting protease enzymes in biological samples, such as wound fluids. The present invention also relates to diagnostic systems comprising such diagnostic devices.

In mammals, injury triggers an organised complex cascade of cellular and biochemical events that result in a healed wound. Wound healing is a complex dynamic process that results in the restoration of anatomic continuity and function; an ideally healed wound is one that has returned to normal anatomic structure, function and appearance.

Infection of wounds by bacteria delays the healing process, since bacteria compete for nutrients and oxygen with macrophages and fibroblasts, whose activities are essential for the healing of the wound. Infection results when bacteria achieve dominance over the systemic and local factors of host resistance. Infection is therefore a manifestation of a disturbed host/bacteria equilibrium in favour of the invading bacteria. This elicits a systemic septic response, and also inhibits the multiple processes involved in wound healing. Lastly, infection can result in a prolonged inflammatory phase and thus slow healing, or may cause further necrosis of the wound. The granulation phase of the healing process will begin only after the infection has subsided. Accordingly, there is a need for rapid, accurate identification of the amount and type of bacteria present in wound fluid, preferably before clinical signs of infection become apparent.

Infected wounds generally produce substantially more exudate than non-infected wounds, and the composition of the wound fluid is different. In particular, it has been found that levels of elastase are elevated in infected wounds, both during and immediately before the onset of clinical signs of wound infection. In addition, each type of infective microorganism produces characteristic protease enzymes.

The amount and composition of wound fluid (exudate) produced by a wound depends on the type of wound and the history of wound healing. For example, surgical wounds have an acute inflammatory phase of a few days during which discharge is significant, after which the rate of exudate production can be expected to fall sharply. Chronic wounds, such as ulcers, produce wound fluid containing elevated levels of endogenous protease enzymes, in particular matrix metalloproteinase (MMP) enzymes including elastase and gelatinase. Burns produce large amounts of wound exudate having characteristic properties. Pain is also associated with characteristic protease enzymes in wound fluid. Biochemically, pain is experienced when there is an increase of kinins (bradykinin) in the area of the wound. Kinins are produced by the proteolytic breakdown of kininogen, and the protease responsible for this is kallikrein.

Accordingly, a need exists for diagnostic devices that can be used to determine the levels of endogenous and/or bacterial protease enzymes in wound fluid in order to provide a physician or care giver with information about the condition of the wound.

WO95/04280 describes lateral flow devices for analysing medical samples, for example samples taken from medical swabs. These devices rely on the use of immunological binding partners to perform immunoassays, such as an enzyme-linked immunosorbent assays (ELISA) within the device. The disadvantages of immunoassays are well known in the art.

WO03/063693 and US2003/0096315 describe methods of diagnosing wound infection based on the detection of enzymes that have been secreted by microorganisms, or expressed on the cell surface of microorganisms, or expressed on the surface of a cell infected with a virus or prion. The enzymes can for example modify labelled substrates, for example proteins or polypeptides, by cleavage, and such modification can be detected to determine the presence or absence of the infective agent. The labelled substrates therefore serve as markers for the detection of the presence or absence of a microorganism in a sample, for example, a wound or body fluid. In certain embodiments, suitable substrates are labelled with at least one color-forming component attached through a peptide linker. The substrates produce a visible color change when the linker is cleaved by the analyte enzyme.

Mitchell C. Sanders et al. in patent application PCT/US2004/028675 entitled SIGNAL AMPLIFICATION USING A SYNTHETIC ZYMOGEN filed on 2 Sep. 2004 describe further methods for the detection of enzymes that have been secreted by microorganisms in a sample of a biological fluid. The methods are based on exposing a zymogen to the biological sample. The term “zymogen” refers to a complex of a signal enzyme with an exogenous peptide that is a substrate for the analyte enzyme. The peptide inhibits the activity of the signal enzyme. The exposure occurs under conditions that will facilitate a modification of the exogenous peptide by the enzyme analyte. The modification includes cleaving the exogenous peptide, and the cleavage results in activation of the signal enzyme and a detectable signal. Suitable signal enzymes include green fluorescent protein (GFP), luciferase, laccase (CotA), and horseradish peroxidase (HRP). In some embodiments, the zymogen is attached to a solid surface by a linker group comprising the exogenous peptide, and the modification includes cleaving the exogenous peptide to detach the signal enzyme from the solid surface. In one embodiment, the testing device comprises a membrane, at least one exogenous peptide attached to the membrane, a signal enzyme attached to the peptide, and at least one detectably labeled substrate attached to the membrane at a second location. Signal enzyme is detached from the membrane by cleavage of the peptide linker by the analyte enzyme, and diffuses or is carried to the second layer where it reacts with the detectably labelled substrate to give a detectable signal.

In a first aspect, the present invention provides a device for detecting a mammalian host-derived enzyme in a sample of a wound fluid, wherein the device comprises: a housing having an inlet for the sample and side walls defining a fluid flow path extending from the inlet, an indicator moiety that is bound to a solid substrate by means of a peptide linker moiety that is cleavable by the host-derived enzyme, the solid substrate being located in a reaction zone of the fluid flow path; and a detector moiety located in a detection zone downstream from the reaction zone in the fluid flow path, wherein the detector moiety can interact with an indicator moiety that has been cleaved from the solid substrate to produce a detectable change in the detection zone.

The term “mammalian host-derived enzyme” refers to an endogenous enzyme found in mammalian wound fluid. The enzyme is normally a protease enzyme. The term “protease enzyme” refers to an enzyme that specifically or non-specifically cleaves a peptide, polypeptide, or protein.

Endogenous (i.e. host-derived) protease enzymes that may be detected by the devices and systems of the present invention are suitably selected from the group consisting of matrix metalloproteinases (MMP's), elastase, stromelysin, kallikrein and thrombin. Suitably, the endogenous protease is selected from the group consisting of neutrophil proteases and macrophage proteases. Preferred protease enzymes include collagenases (e.g. MMP-1 and MMP-8), gelatinases (e.g. MMP-9) and neutrophil elastase, MMP-2, MMP-12, proteinase 3, plasmin, low molecular weight gelatinases and latent or active elastases, interleukin converting enzymes and tumor necrosis factor (TNFα) converting enzymes.

In certain embodiments according to this aspect, the device further comprises a control moiety located in a control zone in said in said device, wherein the control moiety can interact with a component of the wound fluid sample to improve the accuracy of the device.

In a second aspect, the invention provides a device for detecting an analyte enzyme in a sample of a wound fluid, said device comprising: a housing having an inlet for the sample and side walls defining a fluid flow path extending from said inlet; an indicator moiety that is bound to a solid substrate by means of a peptide linker moiety that is cleavable by said analyte enzyme, said solid substrate being located in a reaction zone of said fluid flow path; a detector moiety located in a detection zone downstream from the reaction zone in said fluid flow path, wherein the detector moiety interacts with an indicator moiety that has been cleaved from said solid substrate to produce a detectable change in said detection zone; and a control moiety located in a control zone in the device, wherein the control moiety can interact with a component of the wound fluid sample to improve the accuracy of the device.

In the devices according to this aspect, the analyte enzyme may be selected from the group consisting of mammalian host-derived enzymes (for example as defined above in relation to the first aspect of the invention), and microbial enzymes. The term “microbial enzymes” includes protease enzymes that have been secreted by microorganisms, or expressed on the cell surface of microorganisms such as bacteria or fungi.

Examples of pathogenic bacteria that may be detectable by the present invention include, but are not limited to staphylococcus (for example, Staphylococcus aureus, Staphylococcus epidermidis, or Staphylococcus saprophyticus), streptococcus (for example, Streptococcus pyogenes, streptococcus pneumoniae, or Streptococcus agalactiae), enterococcus (for example, Enterococcus faecalis, or Enterococcus faecium), corynebacteria species (for example, Corynebacterium diptheriae), bacillus (for example, Bacillus anthracis), listeria (for example, Listeria monocytogenes), Clostridium species (for example, Clostridium perfringens, Clostridium tetanus, Clostridium botulinum, Clostridium difficile), Neisseria species (for example, Neisseria meningitidis, or Neisseria gonorrhoeae), E. coli, Shigella species, Salmonella species, Yersinia species (for example, Yersinia pestis, Yersinia pseudotuberculosis, or Yersinia enterocolitica), Vibrio cholerae, Campylobacter species (for example, Campylobacter jejuni or Campylobacter fetus), Helicobacter pylori, pseudomonas (for example, Pseudomonas aeruginosa or Pseudomonas mallei), Peptostreptococcus (for example Peptostreptococcus magnus), Bacteroides fragilis, Haemophilus influenzae, Bordetella pertussis, Mycoplasma pneumoniae, Ureaplasma urealyticum, Legionella pneumophila, Treponema pallidum, Leptospira interrogans, Borrelia burgdorferi, mycobacteria (for example, Mycobacterium tuberculosis), Mycobacterium leprae, Actinomyces species, Nocardia species, chlamydia (for example, Chlamydia psittaci, Chlamydia trachomatis, or Chlamydia pneumoniae), Rickettsia (for example, Rickettsia ricketsii, Rickettsia prowazekii or Rickettsia akari), brucella (for example, Brucella abortus, Brucella melitensis, or Brucella suis), Proteus mirabilis, Serratia marcescens, Enterobacter clocae, Acetinobacter anitratus, Klebsiella pneumoniae and Francisella tularensis.

Suitably, the microbial enzyme comprises a bacterial protease selected from the group consisting of bacterial enzymes of Pseudomonas Aeruginosa, Enterococcus faecalis, Escherichia Coli, Streptococcus Pyogenes and Staphylococcus Aureus. The enzymes are species specific and in some cases the actual enzyme marker being detected is not known.

The above aspects of the present invention share the special technical feature of a laterally spaced reaction zone and detection zone. This provides a compact, “lateral flow” analytical device that does not suffer from the disadvantages of prior art assays. In particular, since it uses a readily synthesised peptide linker in a substrate for the analyte enzyme, the reagents are inexpensive to prepare and stable under normal sterilization and storage conditions. Furthermore, the device detects active enzymes only, and does not give false positives for inactive enzymes or proenzymes.

The fluid flow path of the assay device will typically be capable of supporting lateral flow of aqueous liquids. By “lateral flow”, it is meant liquid flow in which all of the dissolved or dispersed components of the liquid are carried, preferably at substantially equal rates, and with relatively unimpaired flow, laterally through the carrier. Suitably, the fluid flow path contains one or more porous carrier materials. The porous carrier materials are preferably in fluid communication along substantially the whole fluid flow path so as to assist transfer of fluid along the path by capillary action. Suitably, the porous carrier materials are hydrophilic, but preferably they do not themselves absorb water. The porous carrier materials may function as solid substrates for attachment of the indicator moieties in the reaction zone and/or the detector moieties in the detection zone.

Suitable materials for forming the porous carrier materials include any suitable natural or synthetic polymer, including insoluble polysaccharides such as cellulose, and synthetic polymers such as polyacrylates, high density porous polyethylene sheet materials, polyvinyl chloride, polyvinyl acetate, copolymers of vinyl acetate and vinyl chloride, polyamide, polycarbonate, nylon, glass fiber, orlon, polyester, polystyrene, and mixtures and combinations thereof. A suitable material is POREX (Registered Trade Mark) lateral flow membrane, available from Porex Technologies Corp., Fairburn Ga., USA. Suitably, the carrier material comprises pendant amine, ester or carboxylate groups to assist conjugation.

The size and shape of the carrier are not critical and may vary. The carrier defines a lateral flow path. Suitably, the porous carrier is in the form of one or more elongate strips or columns. In certain embodiments, the porous carrier is one or more elongate strips of sheet material, or a plurality of sheets making up in combination an elongate strip. The reaction zone and detection zone would then normally be spaced apart along the long axis of the strip. However, in some embodiments the porous carrier could, for example be in other sheet forms, such as a disk. In these cases the reaction zone and detection zone would normally be arranged concentrically around the center of the sheet, with a sample application zone in the center of the sheet. In yet other embodiments, the carrier is formed of carrier beads, for example beads made from any of the materials described above. The beads may suitably be sized from about 1 micrometer to about 1 mm. The beads may be packed into the flow path inside the housing, or may be captured or supported on a suitable porous substrate such as a glass fiber pad.

Normally, the fluid flow path further comprises a sample application zone upstream of the reaction zone and in fluid communication with the inlet of the housing. The device is configured such that, in use, the fluid sample flows laterally from the sample application zone to the reaction zone and then to the detection zone.

It will be appreciated that the devices according to the present invention may be adapted to detect more than one enzyme analyte. This can be done by the use of several different peptide linkers in a single reaction zone, or preferably by the provision in a single device of a plurality of lateral flow paths each having a different peptide linker group in its respective reaction zone for detecting a different enzyme. In certain embodiments, the plurality of lateral flow paths are defined as separate fluid flow paths in the housing, for example the plurality of lateral flow paths may be radially distributed around a sample receiving port. In some embodiments, the plurality of fluid flow paths are physically separated by the housing. In other embodiments multiple lateral flow paths (lanes) can be defined in a single lateral flow membrane by depositing lines of wax or similar hydrophobic material between the lanes.

The housing is normally made of a thermoplastic material such as polyethylene, polypropylene, polyacrylate, polyamide or polystyrene. Suitably, the housing is made by fitting together two plastic components forming the top and bottom of the device, respectively. Suitable methods for making the housing components include injection molding and stereolithography. Suitably, the housing is at least partially transparent. This allows visible diagnostic indicators to be observed through the housing. In some embodiments the housing is at least partially transparent to ultraviolet light, for example light having wavelength 300-350 nanometers. In some embodiments, the housing comprises a window for observing the detection zone.

The cleavable peptide linkers generally comprise cleavable oligopeptidic sequences, each typically of twenty residues or fewer, for example from 3 to 15 residues.

The sensitivity of the diagnostic device will depend on a number of factors, including the length of the cleavable linker sequences. Steric hindrance may also be reduced by coupling the cleavable oligopeptidic sequence to the substrate by means of an appropriate spacer. Thus, the oligopeptidic sequences may couple the signal moiety directly to the substrate (in which case the cross-linkage consists of the oligopeptidic sequence) or by means of an appropriate spacer. Suitable conjugation methods incorporating spacers are described in U.S. Pat. No. 5,770,229.

The following paper gives a useful review of bioconjugation techniques for use in pharmaceutical chemistry: Veronese, F. M. and Morpurgo, M (1999) Bioconjugation in Pharmaceutical chemistry II Farmaco, 54, 497-516 and Ulbrich, K., et al (2000) Journal of controlled release 64, 63-79. The entire contents of these papers are hereby incorporated by reference.

As already noted, the endogenous (host-derived) proteases to be detected may include elastase. In such embodiments, suitable substrate linkers may include one or more of the oligopeptidic sequences Lys-Gly-Ala-Ala-Ala-Lys-Ala-Ala-Ala-, Ala-Ala-Pro-Val, Ala-Ala-Pro-Leu, Ala-Ala-Pro-Phe, Ala-Ala-Pro-Ala or Ala-Tyr-Leu-Val.

In certain embodiments, the host-derived proteases to be detected may include a matrix metalloproteinase, in particular MMP-2 or MMP-9. In these embodiments, the cleavable linker may comprise the oligopeptidic sequence -Gly-Pro-Y-Gly-Pro-Z-, -Gly-Pro-Leu-Gly-Pro-Z-, -Gly-Pro-Ile-Gly-Pro-Z-, or-Ala-Pro-Gly-Leu-Z-, where Y and Z are amino acids.

In certain embodiments, the host-derived proteases to be detected may include a collagenase. In these embodiments, the cleavable linker may comprise the oligopeptidic sequence -Pro-Leu-Gly-Pro-Z-Arg-Z-, -Pro-Leu-Gly-Leu-Leu-Gly-Z-, -Pro-Gln-Gly-Ile-Ala-Gly-Trp-, -Pro-Leu-Gly-Cys-His-, -Pro-Leu-Gly-Leu-Trp-Ala-, -Pro-Leu-Ala-Leu-Trp-Ala-Arg-, or -Pro-Leu-Ala-Tyr-Trp-Ala-Arg-, where Z is an amino acid.

In certain embodiments, the host-derived proteases to be detected may include a gelatinase. In these embodiments, the cleavable linker may comprise the oligopeptidic sequence -Pro-Leu-Gly-Met-Trp-Ser-Arg-.

In certain embodiments, the host-derived proteases to be detected may include thrombin. In these embodiments, the cleavable linker may comprise the oligopeptidic sequence -Gly-Arg-Gly-Asp-, -Gly-Gly-Arg-, -Gly-Arg-Gly-Asp-Asn-Pro-, -Gly-Arg-Gly-Asp-Ser-, -Gly-Arg-Gly-Asp-Ser-Pro-Lys-, -Gly-Pro-Arg-, -Val-Pro-Arg-, or-Phe-Val-Arg-.

In certain embodiments, the host-derived proteases to be detected may include stromelysin. In these embodiments, the cleavable linker may comprise the oligopeptidic sequence -Pro-Tyr-Ala-Tyr-Trp-Met-Arg-.

In certain embodiments, the host-derived proteases to be detected may include a kallikrein. The term “a kallikrein” refers to all serine proteases, whose activation is associated with the degradation of kininogen to form kinins, which are implicated in the onset of pain. Suitable peptide sequences for use in cleavable substrates for kallikrein include -Phe-Arg-Ser-Ser-Arg-Gln- or -Met-Ile-Ser-Leu-Met-Lys-Arg-Pro-Gln- that can be degraded by kallikrein at Lys-Arg or Arg-Ser bonds.

In embodiments according to the second aspect of the present invention, the peptide linkers may be cleavable by a microbial enzyme. The peptide linkers for the detection of specific microbial protease enzymes are determined using a variety of techniques including literature references to the natural substrate of the enzyme and empirical screening. Methods of identifying bacterial enzymes and peptide substrates therefor are described in WO03/063693, US-A-2003/0096315 and WO2004/047614, the entire contents of which are incorporated herein by reference. Suitable peptide linkers include the following:

| | |
|---|---|
| Sequence 1: | ETKVEENEAIQK |
| Sequence 2: | VTLENTALARC |
| Sequence 3: | QADALHDQASALKC |
| Sequence 4: | KVSRRRRRGGDKVSRRRRRGGD |

Sequences 1 and 2 above are specific for Staphylococcus, Sequence 3 is specific for Pseudomonas, Sequence 4 is specific to E. coli.

In some embodiments, the peptide is a sequence having at least 50%, 60%, 70%, 80%, 85%, 90%, 95%, or 99% sequence identity to one of the sequences listed herein, as determined using a sequence comparison program and parameters described herein.

Suitably, the indicator moiety comprises an indicator enzyme, and the detection zone contains a reagent that undergoes a visible change in the presence of the indicator enzyme. For example, the indicator enzyme may be a redox enzyme. Suitable indicator enzymes include laccase (CotA), green fluorescent protein (GFP), luciferase, alkaline phosphatase (ALP), p-galactosidase, acetylcholinesterases, and in particular horseradish peroxidase (HRP).

The detector moiety in the detection zone preferably undergoes a color change (the term color change includes chemiluminescence and/or a change in appearance under UV light) in the presence of one or more of the indicator moieties, and preferably this color change is visible through the window or side wall of the housing. The color change may be assessed either visually (e.g. by comparison with a color key) or by optical detection devices, such as reflectance analyzers, video image analyzers and the like. Thus, target analyte levels may be determined by devices of the present invention.

Suitably, the indicator moity is a redox enzyme, and in these embodiments the reagent in the detection zone may then be a redox indicator. For example, where the indicator moiety is HRP, the indicator moiety may comprise 4-chloro-1-naphthol, which undergoes a color change from colorless to blue in the presence of HRP and hydrogen peroxide. It is also possible that other HRP substrates could be used. These would include TBM (3,3,5,5-tetramethylbenzidine), DAB (3,3-Diaminobenzidine), DAB with metal enhancement such as cobalt and nickel, AEC (3-amino-9 ethylcarbazole), or a combination of these substrates.

When the indicator moiety comprises alkaline phosphatase, then the detector moiety may comprise BCIP/NBT or p-nitrophenylphosphate. When the indicator moiety comprises CotA, the detector moiety may comprise naphthol, which changes from colorless to dark blue in the presence of CotA. When the indicator moiety comprises luciferase, the detector moiety may comprise luciferin, which undergoes a chemiluminescent reaction with luciferase.

An absorbent element is suitably included in the devices of the present invention. The absorbent element is located in the fluid flow path downstream from the detection zone. The absorbent element is a means for drawing the whole sample through the reaction zone and the detection zone by capillary attraction. Generally, the absorbent element will consist of a hydrophilic absorbent material such as a woven or nonwoven textile material, a filter paper or a glass fiber filter.

The device may further comprise at least one filtration element to remove impurities from the sample before the sample contacts the reaction zone. The filtration device may for example comprise a microporous filtration sheet for removal of cells and other particulate debris from the sample. The filtration device is typically provided upstream of the sample application zone of the fluid flow path, for example in the inlet of the housing or in the housing upstream of the inlet. The filtration device is preferably omitted when the analyte enzyme is a cell wall enzyme, for example a cell wall protease is a preferred analyte for the detection of E. coli.

Preferably, the devices according to the present invention include a control moiety in a control zone of the device, wherein the control moiety can interact with a component of the wound fluid sample to improve the accuracy of the device. The control zone may be in the same fluid flow path as the reaction and detection zones, or it may be in a different fluid flow path in the device.

Suitably, the control zone is adapted to reduce false positive or false negative results. A false negative result could arise for various reasons, including (1) not all of the sample has passed through the reaction zone into the detection zone, or (2) the sample was too small to start with. In order to address false negative mechanism (1), the control zone may comprise an end-of-assay indicator to indicate when a predetermined minimum volume of liquid has passed through the reaction zone.

For example, the end of assay indicator may consist of a pH indicator associated with the absorbent zone at the downstream end of the device. Upon contact with the sample, a pH change occurs in the processed absorbent. This pH shift converts the pH indicator to a different color, which is seen in an observation window over the absorbent zone. Alternatively, the end of assay indicator may comprise a line of soluble ink downstream of the detection zone. The dissolution of this line of soluble ink will then signify that a sufficient quantity of liquid has passed through the detection zone.

In order to address false negative mechanism (2), the control zone preferably further comprises a reference assay element for determining the total protease content or the total protein content of the sample, that is to say for establishing that the total protease content or the total protein content of the sample is higher than a predetermined minimum. It is possible to indicate the presence of protein by placing a line of tetrabromophenol blue downstream from the detection zone. This line will change from colorless to blue depending on the concentration of protein present. It is also possible to detect glucose (using glucose oxidase), blood (using diisopropyl-benzene dihydro peroxide and tetramethylbenzidine), leukocytes (using ester and diazonium salt). These may all be useful analytes for detection in the control zone for the reduction of false negatives.

False positive readings can arise due to the presence of unbound indicator moiety in the reagent zone, and/or due to the presence of sub-pathological levels of the protease analyte in the sample. In order to reduce these false positives, the control zone preferably further comprises a buffer reagent positioned in the lateral flow path intermediate the reaction zone and the detection zone. The buffer reagent is adapted to absorb or otherwise inactivate a predetermined threshold amount of the indicator moiety to prevent false positive readings. Only when the amount of indicator released from the reaction zone exceeds the threshold amount can the excess indicator moiety pass through the saturated buffer and into the detection zone to give a positive signal. A suitable buffer control zone may be provided by placing a line, or several lines, of a substance which may capture the indicator moiety (eg. Antibodies to the moiety). A suitable buffer zone for a device that uses HRP as the signal enzyme is a zone comprising nitrocellulose in the flow path upstream of the detection zone. For example, such a buffer zone may be provided by depositing a line of a solution of nitrocellulose in an organic solvent on the substrate material, followed by evaporation of the solvent.

False positives can also arise from the presence of interfering enzymes. That is to say, enzymes other than the analyte enzyme that can cleave the exogenous peptide sequence in the reaction zone. Endogenous elastase enzymes can be troublesome interfering enzymes in some assays. This problem can for example be addressed by providing a control moiety in a control zone upstream of the reaction zone, wherein the control moiety comprises a binding partner for the interfering enzymes that is bound to a solid substrate in the control zone, whereby the interfering enzymes are captured in the control zone.

Alternatively or additionally, the devices, systems or kits of the present invention may incorporate the specific binding/inhibiting agents into a wash buffer or other sample pretreatment feature.

In a further aspect, the present invention provides a diagnostic test system or kit comprising a diagnostic device according to the present invention. The test system or kit may comprise, in addition to a diagnostic device according to the present invention, one or more components selected from: a color chart for interpreting the output of the diagnostic device, a sampling device for collecting a sample of a wound fluid from a wound, a wash liquid for carrying a sample of wound fluid through the lateral flow device, and a pretreatment solution containing a reagent for pretreatment of the wound fluid sample.

Where present, the sampling device may comprise a swab or a biopsy punch, for example a shaft having a swab or biopsy punch attached thereto. Suitably, the housing of the diagnostic device includes a sample receiving port in fluid communication with the inlet to the fluid flow path, and preferably the housing and the swab or biopsy punch comprise complementary fitting elements whereby the swab or biopsy punch can be secured to the housing with the swab or biopsy punch received in the sample receiving port.

In certain embodiments the fitting element on the shaft may be located from 1 mm to about 30 mm from the base of the swab or the biopsy punch. This is consistent with the use of relatively small sample receiving port on the housing of the diagnostic device. The sample receiving port is typically located on an upper surface of the diagnostic device, and it is typically generally in the form of an upwardly projecting tube, open at the top and having the inlet to the fluid flow path located at the bottom of the tube. Suitable swabs, biopsy punches and sample receiving caps are described in detail in copending applications GB0403976.4 and GB0403978.0 both filed on 23 Feb. 2004, the entire contents of which are incorporated herein by reference.

The fitting element on the shaft may a tapered region of the shaft for forming an interference fit with the housing, for example it may appear as a truncated cone that is coaxial with the shaft and tapers towards the first end of the shaft. Or the whole shaft may have a diameter larger than that of the swab or biopsy punch, with a tapered region adjacent to the first end. In any case, the diameter of the tapered region where it engages with the housing is normally greater than the diameter of the swab or biopsy punch, so that the inlet port can enclose the swab or biopsy punch.

In other embodiments, the engagement element may comprise a snap-fitting projection for forming a snap-fit with one or more complementary projections on an inner surface of the housing, or a threaded projection for forming a screw fit with one or more complementary threads on an inner surface of the cap, or a Luer-lock type fitting.

The swab may be any absorbent swab, for example a nonwoven fibrous swab. Typically the diameter of the swab is about 2 to about 5 mm, for example about 3 mm. In certain embodiments, the swab may be formed from a medically acceptable open-celled foam, for example a polyurethane foam, since such foams have high absorbency and can readily be squeezed to expel absorbed fluids. The biopsy punch will typically be a stainless steel cylindrical punch of diameter about 1 mm to about 10 mm, for example about 3 mm to about 8 mm, suitably about 6 mm.

In certain embodiments the shaft is hollow, whereby a fluid can be passed down the shaft from the second end to expel the biological sample from the swab or the biopsy punch into the diagnostic device. This helps to ensure that all of the sample passes through the porous carrier, thereby avoiding false negatives. The shaft may comprise a fitting at the second end for attachment of a syringe or other source of the fluid. In certain embodiments, the apparatus may comprise a reservoir of liquid attached to the second end of the shaft, for example a compressible bulb containing the liquid, which can be activated after use of the swab or biopsy punch. Suitable devices of this kind are described, for example in U.S. Pat. No. 5,266,266, the entire content of which is incorporated herein by reference. In other embodiments, the apparatus may comprise a plunger that can be pushed down the hollow bore of the shaft to expel fluid or other specimens from the swab or biopsy punch.

Another advantage of the hollow shaft is that, where the apparatus is a biopsy punch, the biopsy sample can more readily be pushed or blown out of the punch. The biopsy punch apparatus can further comprise a homogenizing tool that can be passed down the hollow shaft to homogenize a tissue sample in the biopsy punch. This step of homogenizing can be followed, if necessary, by passing liquid down the shaft from the second end to expel the homogenized tissue from the biopsy punch into the device for diagnostic analysis.

In this aspect of the invention, the swab or biopsy punch may be sterilized, and may be packaged in a microorganism-impermeable container. The diagnostic devices according to the present invention may also be sterilized, but they may not, because the devices often do not come into contact with the patient being diagnosed.

The devices and systems of the present invention provide a means for one-step detection of analytes. Generally, the samples will be biological material obtained or derived from patients. Physiological materials, such as urine, serum, cerebrospinal fluid, gastric secretions, nasal secretions, sputum, pharyngeal exudates, urethral or vaginal secretions, and the like may be assayed directly to detect the presence of analytes. Alternatively, when assaying tissue samples, the tissue will often require dissociation or liquification prior to insertion into devices of the present invention. In some instances it may be desirable to dilute the sample prior to performing the assay. Alternatively, analytes in the sample may be concentrated as by filtration or centrifugation. Preferably, the sample is a sample of wound fluid or a biopsy sample (suitably macerated or extracted) from a wound.

The term “wound fluid” refers to any wound exudate or other fluid (preferably substantially not including blood) that is present at the surface of the wound, or that is removed from the wound surface by aspiration, absorption or washing. The measuring is preferably carried out on wound fluid that has been removed from the body of the patient, but can also be performed on wound fluid in situ. The term “wound fluid” does not normally refer to blood or tissue plasma remote from the wound site.

Any type of wound may be diagnosed for treatment using the devices and systems of the present invention. For example, the wound may be an acute wound such as an acute traumatic laceration, perhaps resulting from an intentional operative incision. More usually the wound may be a chronic wound. Preferably, the chronic wound is selected from the group consisting of venous ulcers, pressure sores, decubitis ulcers, diabetic ulcers and chronic ulcers of unknown aetiology. Chronic wound fluids typically have levels of markers such as neutrophil elastase that are many times the level found in normal, acute wound fluids. Nevertheless, it has been found that the levels of such markers increase still further in infected chronic wounds.

Specific embodiments of the present invention will now be described further, by way of example, with reference to the accompanying drawings, in which:

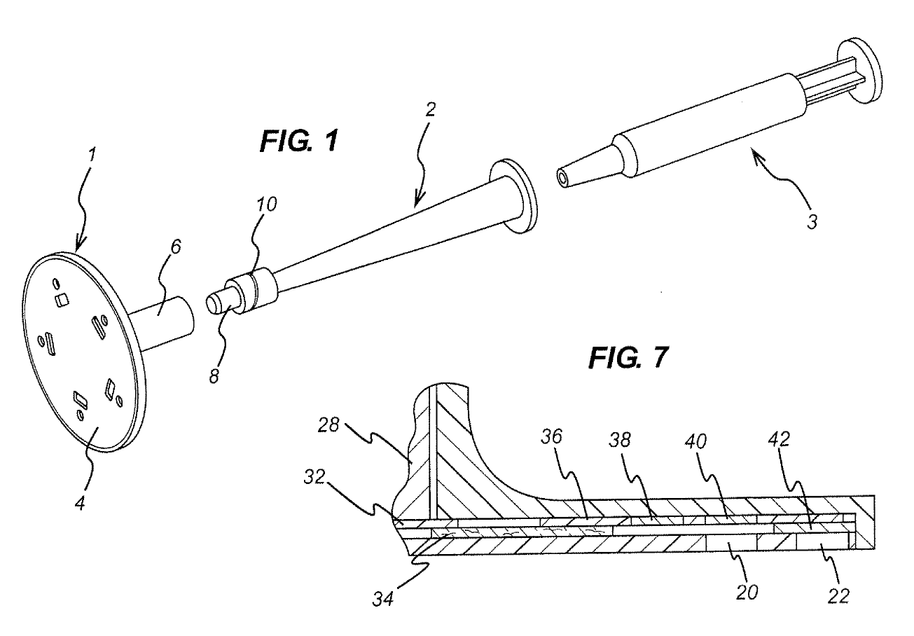

FIG. 1 is a perspective view of a diagnostic device and swab system according to the present invention;

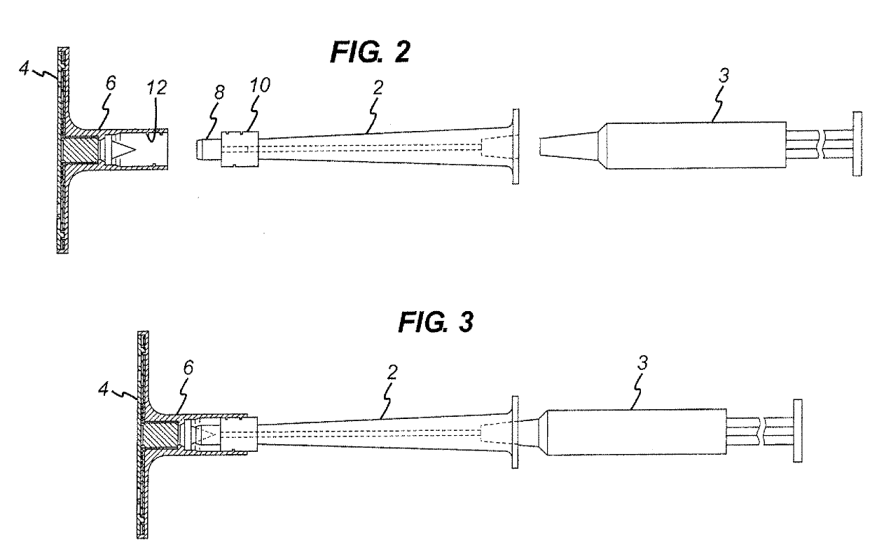

FIG. 2 is a longitudinal cross-section through the system of FIG. 1 before insertion of the swab into the sample receiving port of the device;

FIG. 3 is a longitudinal cross-section through the system of FIG. 1 after insertion of the swab into the sample receiving port of the device, and during the analysis using the device;

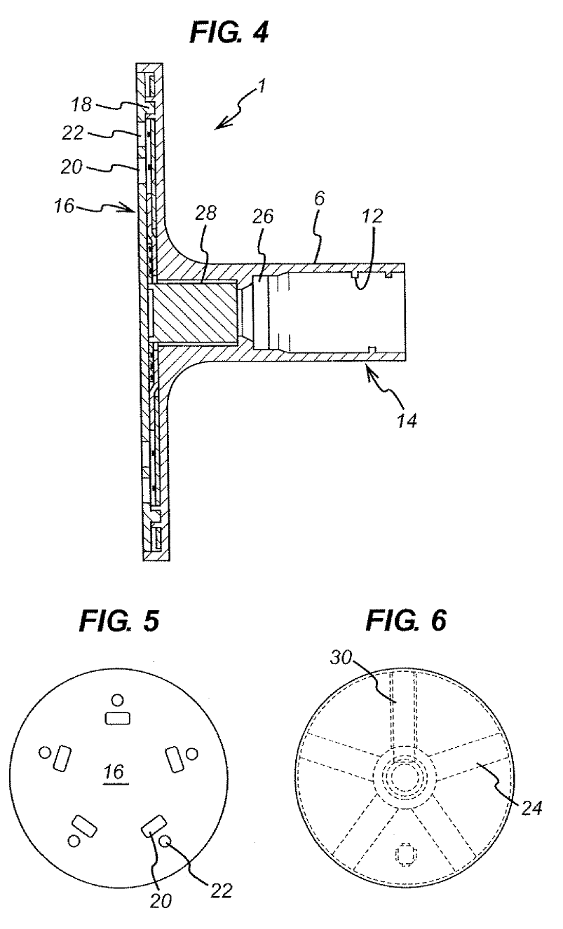

FIG. 4 is a more detailed longitudinal cross sectional view through the analysis device of FIG. 1;

FIG. 5 is a bottom plan view of the analysis device of FIG. 1;

FIG. 6 is a bottom plan view of the analysis device of FIG. 1 with the bottom housing part removed; and

FIG. 7 is a schematic partial cross-section through one of the fluid flow paths in a device according to the present invention, similar to that of FIG. 1, showing the different zones of the fluid flow path.

Referring to FIGS. 1 to 4, the assay system comprises a protease detection device 1, a swab 2 and a syringe 3. The protease detection device 1 is generally disc-shaped, with a tubular sample receiving port 6 projecting upwardly from the center of the disc in a generally cylindrical formation. The swab 2 comprises a collection sponge 8 of open-celled hydrophilic polyurethane foam, and a shaft 2 of injection-molded thermoplastic. The shaft 2 is provided at its lower end proximate to the sponge 8 with a radially enlarged region 10 that forms a substantially fluid-tight engagement inside the tubular receiving port 6 of the device 1 by means of luer lock fittings on the enlarged region 10 and the inside surface 12 of the sample receiving port 6.

The shaft 2 of the swab is hollow. The syringe 3 contains a predetermined volume of sterile saline solution for injection down the hollow shaft to expel the sample from the sponge 8 into the detection device, and to carry the sample through the fluid flow channel of the device. In certain embodiments, the solution contains a binding partner or other inactivating agent for one or more interfering enzymes or other factors that may be present in the sample.

Referring to FIG. 4, the device 1 comprises a housing formed from an upper part 14 and a substantially disc-shaped lower part 16. Each part may be made by injection molding of thermoplastics. The upper housing part is itself made from two separate pieces. The parts are fitted together by means of snap fittings 18. The lower surface of the upper housing part 14 comprises five radially spaced shallow recesses 24 for receiving respective test strips 30 of porous carrier materials, as shown in FIG. 6. Each test strip is adapted to test for a different protease enzyme characteristic of a different wound pathology. The test strips are retained in the recesses by the lower housing part 16. The fluid flow paths for the samples extend between the upper and lower housing parts along the recesses 24 when the lower housing part 16 and the upper housing part 14 are snap-fitted together.

As already noted, a tubular inlet port 6 for receiving the swab projects upwardly from the upper housing part 14. A filter membrane can be located in the bottom of the inlet port in region 26 for removing particles and cells from the sample, but in this embodiment the membrane is omitted in order to allow free passage to cell surface enzyme analytes. The sample then flows through a cylindrical (annular) inlet channel 28 to the upstream ends of the five test strips 24.

The lower housing part 16 comprises apertures for viewing the test strips. For each of the radially disposed test strips there is a detection window 20 above the detection zone of the strip, and a control window 22 above the end-of-assay indicator region of the test strip. The lower surface of the second housing part may further be provided with embossing or other indicia identifying the specific protease or pathogen detected in that strip. It will be appreciated that the windows 20,22 and optional indicia could alternatively be provided in the upper housing part 14 if viewing from the top of the device is preferred.

Referring to FIG. 7, a schematic, enlarged cross-section perpendicular to one of the lateral flow paths of a device similar to that of FIG. 1 is shown. The flow path in this device contains a number of elements retained by the plastic housing. The first element in the flow path is a wicking pad 32 of non-woven textile material. This is located at the radially innermost end of the flow path, and positioned directly under the inlet channel 28 of the device. The wicking pad 32 is in capillary contact with reaction zone 34 of the flow path.

Reaction zone 34 is a porous glass fiber pad approximately 1 mm thick, 1 cm wide, and 3 cm long (i.e. extending about 3 cm radially outward from the inlet 28). The glass fiber pad incorporates support beads having horseradish peroxidase (HRP) conjugated thereto by a peptide-containing linker as hereinbefore described. The peptide has 10-15 residues and contains the sequence ala-ala-pro-val that is specific to elastase.

Conjugation of HRP to the substrate-bound peptides can for example be achieved through the bi-functional crosslinking reagent sulfo-SMCC. The reaction is a two step process, including 1) formation of HRP-maleimide, followed by 2) reaction with a peptide that has been conjugated to the substrate surface using conventional methods.

Typical conditions for the HRP Conjugation as described by Mitchell C. Sanders et al. (op.cit.) are: (1) HRP—Maleimide conjugation: 2.5 mg of Roche HRP is dissolved in 500 μl of 1M Na phosphate (pH of 7.4). Sulfo-SMCC is dissolved in 50 μl DMSO, and combined with the HRP for 20 minutes at room temperature. Separation is accomplished with Gel filtration in maleimide conjugation buffer; (2) HRP-Peptide-Membrane Conjugation: The fractions that are free of sulfo-SMCC (˜1 ml) are combined with the substrate-bound peptide and reacted at ˜4° C. overnight with rotation. Several washes are conducted, including two 20-minute washes with 100 mL of 0.1% triton in PBS, followed by two 20-minute washes with 0.1% PEG 5000 solution, and a 1 hr wash in 250 mL of a 10% sucrose solution, followed by speed vacuuming overnight.

An aliquot containing 10 μl of a 10% solution of the conjugated beads is then pipetted onto the glass fiber strip to form the reaction zone.

The radially outermost end of the reaction zone pad 34 is in capillary contact with detection strip 36. Detection strip 36 is formed of a POREX membrane of total length about 4 cm having deposited thereon a detection region 40 of 4-chloro-1-naphthol that undergoes a color change in the presence of HRP. The detection zone 40 is made by depositing 0.5 μl of a 40 mg/ml solution of 4-chloro-1-naphthol in ethanol onto a 1 cm region of the detection strip.

The detection strip 36 is further provided with a control line 38 of nitrocellulose that reacts with a predetermined threshold amount of free HRP to reduce the incidence of false positive results. To form the control line 38, a solution of 2% nitrocellulose in methanol is made up and 0.5 ul is deposited on a 1 cm line, 1 cm away from the test line.

Finally, the radially outward end of the detection strip 36 is in capillary contact with absorbent reservoir 42, which acts to draw the liquid sample through the test strip and capture the liquid at the end of the strip. The absorbent reservoir 42 is a nonwoven rayon/polyacrylate strip of length about 3 cm that has been impregnated with a solution of 1% tetrabromophenol blue in distilled water and then dried. The indicator in the absorbent reservoir 42 changes color when it is soaked in the test solution, and this color change can be seen through window 22 of the housing. The color change can be used to confirm that a predetermined minimum volume of liquid sample has passed through the detection zone, thereby reducing the likelihood of false negative results.

In alternative embodiments, the swab may be replaced by a biopsy punch, optionally with means to macerate the sample.

In use, the swab is used to obtain a sample of wound fluid. Typically the sample will have volume about 100 ul of wound fluid. The swab is inserted into the sample receiving port of the device by means of the Luer lock. The sample is then expelled from the swab by injecting saline (sterile PBS) through the central channel of the swab from the syringe. The sample passes through the inlet channels and along the test strips. After a predetermined time, the underside of the device is viewed to assess the results of the analysis.

The above embodiments have been described by way of example only. Many other embodiments falling within the scope of the accompanying claims will be apparent to the skilled reader.

1. A device for detecting a mammalian host-derived or an analyte enzyme in a sample of a wound fluid, said device comprising:
a housing having an inlet for the sample and side walls defining a fluid flow path extending from said inlet
an indicator moiety that is bound to a solid substrate by means of a peptide linker moiety that is cleavable by said host-derived enzyme, said solid substrate being located in a reaction zone of said fluid flow path; and
a detector moiety located in a detection zone downstream from the reaction zone in said fluid flow path, wherein the detector moiety interacts with an indicator moiety that has been cleaved from said solid substrate to produce a detectable change in said detection zone.

2. The device according to claim 1, wherein said mammalian host-derived enzyme is selected from the group consisting of neutrophil elastase, matrix metalloproteinases (including the group consisting of MMP-9, MMP-8, MMP-1, MMP-2 and MMP-12), proteinase 3, plasmin, low molecular weight gelatinases and latent or active elastases, interleukin converting enzymes, tumor necrosis factor (TNFα) converting enzymes, and mixtures thereof

3. The device according to claim 1, wherein the device further comprises a control moiety located in a control zone in said device, wherein the control moiety can interact with a component of the wound fluid sample to improve the accuracy of the device.

4. The device according to claim 1, wherein the device further comprises
a control moiety located in a control zone in said device, wherein the control moiety can interact with a component of the wound fluid sample to improve the accuracy of the device.

5. The device according to claim 4, wherein said enzyme is selected from the group consisting of mammalian host-derived enzymes and bacterial enzymes.

6. The device according to claim 5, wherein said enzyme comprises a bacterial enzyme selected from the group consisting of bacterial protease enzymes of Pseudomonas Aeruginosa, Enterococcus faecalis, Escherichia Coli, Streptococcus Pyogenes and Staphylococcus Aureus.

7. (canceled)

8. The device according to claim 1, wherein the fluid flow path contains one or more porous carrier materials.

9. The device according to claim 1, wherein the fluid flow path comprises a sample application zone in fluid communication with said inlet, and the reaction zone is located downstream from the sample application zone.

10. The device according to claim 9, wherein the fluid flow path contains a hydrophilic, porous wicking pad in said sample application zone.

11. The device according to claim 1, wherein the fluid flow path contains an absorbent reservoir of a porous hydrophilic material at the downstream end of the fluid flow path.

12. The device according to claim 1, wherein the indicator moiety comprises an indicator enzyme, and said detector moiety comprises a substrate for the indicator enzyme.

13. The device according to claim 1, wherein the device further comprises at least one filtration element to remove impurities from the sample before the sample contacts the reaction zone.

14. The device according to claim 1, wherein the device comprises an end-of-assay indicator to indicate when a predetermined minimum volume of liquid has passed through the reaction zone.

15. The device according to claim 1, wherein the device comprises a reference assay element for establishing that the total protease content or the total protein content of the sample is higher than a predetermined minimum.

16. The device according to claim 1, wherein the device comprises a buffer zone in the flow path upstream of the detection zone and downstream of the reaction zone, wherein the buffer zone is adapted to bind or inactivate a predetermined minimum amount the detector moiety.

17. The device according to claim 1, wherein the device comprises a control zone in the flow path upstream of the reaction zone, wherein the control zone is adapted to bind or inactivate one or more interfering enzymes.

18. The device according to claim 1, wherein the device comprises a plurality of different said peptide linkers for detecting a plurality of different analyte enzymes.

19. The device according to claim 18, wherein the device comprises a plurality of fluid flow paths each having a different peptide linker group in its respective reaction zone for detecting a different enzyme.

20. The device according to claim 19, wherein said plurality of fluid flow paths are radially distributed around a single sample receiving port.

21. (canceled)

22. (canceled)


SEQUENCE LISTING (4 sequences)

SEQ ID NO: 1 - protein, 12 aa, Staphylococcus
ETKVEENEAIQK

SEQ ID NO: 2 - protein, 11 aa, Staphylococcus
VTLENTALARC

SEQ ID NO: 3 - protein, 14 aa, Pseudomonas
QADALHDQASALKC

SEQ ID NO: 4 - protein, 22 aa, E. coli.
KVSRRRRRGGDKVSRRRRRGGD